(19) United States
(12) Patent Application Publication
(10) Pub. No.: US 2025/0336499 A1
(43) Pub. Date: Oct. 30, 2025

(54) Advice Apparatus, Advice Method, Advice Program, and Advice System

(71) Applicant: SHIMADZU CORPORATION, Kyoto-shi (JP)

(72) Inventors: Mikihiro YAMANAKA, Kyoto-shi (JP); Yutaro NAKATSU, Kyoto-shi (JP); Toshiaki NAKAMURA, Kyoto-shi (JP); Daisuke KAGAMI, Kyoto-shi (JP); Kuniko YOSHIMI, Kyoto-shi (JP)

(21) Appl. No.: 18/873,515

(22) PCT Filed: Apr. 25, 2023
(86) PCT No.: PCT/JP2023/016212

§ 371 Date: Mar. 26, 2025

(30) Foreign Application Priority Data
Jun. 15, 2022 (JP) 2022-096167

(51) Int. Cl. G16H 20/90

(52) U.S. Cl. CPC G16H 20/90

(57) ABSTRACT

An advice device comprises a computing device and a communication device communicably connected to the computing device. The communication device obtains a measurement value of an advanced glycation end product of a subject and a plurality of other data of the subject that are different from the measurement value of the advanced glycation end product. The computing device generates advice information for the life of the subject based on the measurement value of the advanced glycation end product obtained by the communication device and at least one of the plurality of other data obtained by the communication device.

### TECHNICAL FIELD

The present disclosure relates to an advice device, an advice method, an advice program, and an advice system that provide advice for life.

### BACKGROUND ART

One of substances responsible for aging is advanced glycation end products (hereinafter also referred to as “AGEs”). Advanced Glycation End products collectively refer to several compounds, which are formed when sugars combine with proteins and react by oxidation, condensation, and dehydration. AGEs are believed to accumulate in the body as a result of disorders of lifestyle habits such as dietary habits, exercising habits, sleeping habits, inflammation caused by fever or injury, and stress, causing lifestyle-related diseases (e.g., diabetes, dementia, etc.) and age-related diseases.

PTL 1 discloses a sensor which receives fluorescence excited by light radiated to expose the skin of a subject thereto and measures a degree of accumulation of AGEs based on the intensity of the received fluorescence.

### CITATION LIST

### Patent Literature

PTL 1: Japanese Patent Laying-Open No. 2015-223431

### SUMMARY OF INVENTION

### Technical Problem

Conventionally, when a subject has AGEs excessively accumulated and a measurement value of AGEs is evaluated below a reference, a supporter for the subject may provide advice to the subject to improve his/her lifestyle habits. However, if the supporter attempts to provide advice based only on the measurement value of AGEs, the supporter may not be able to specifically determine what type of lifestyle habit the subject should improve, and the supporter may not be able to provide optimal advice to the subject. On the other hand, if the supporter provides advice comprehensively about generally known lifestyle habits in general, there is a possibility that the supporter may unintentionally deny a lifestyle habit that the subject is already working on improving it. This may reduce the subject's positive motivation to improve the lifestyle habit and the subject may be significantly unwilling to accept the advice.

The present disclosure has been made to solve such a problem and contemplates a technique for providing optimal advice to review lifestyle habits.

### Solution to Problem

According to an aspect of the present disclosure, an advice device is an advice device that provides life-related advice. The advice device comprises a computing device and a communication device communicably connected to the computing device. The communication device is configured to obtain a measurement value of an advanced glycation end product of a subject and a plurality of other data of the subject that are different from the measurement value of the advanced glycation end product. The computing device is configured to generate advice information for a life of the subject based on the measurement value of the advanced glycation end product and at least one of the plurality of other data obtained by the communication device.

According to another aspect of the present disclosure, an advice method is a method for providing life-related advice by a computing device. The advice method comprises as a process performed by the computing device: obtaining a measurement value of an advanced glycation end product of a subject and a plurality of other data of the subject that are different from the measurement value of the advanced glycation end product; and generating advice information for a life of the subject based on the measurement value of the advanced glycation end product and at least one of the plurality of other data obtained in the step of obtaining.

According to another aspect of the present disclosure, an advice program is a program that provides life-related advice. The advice program causes a computing device to perform: obtaining a measurement value of an advanced glycation end product of a subject and a plurality of other data of the subject that are different from the measurement value of the advanced glycation end product; and generating advice information for a life of the subject based on the measurement value of the advanced glycation end product and at least one of the plurality of other data obtained in the step of obtaining.

According to another aspect of the present disclosure, an advice system is a system that provides advice for life. The advice system comprises a measurement device, an advice device, and a display device. The measurement device measures an advanced glycation end product of a subject. The advice device generates advice information for a life of the subject based on a measurement value of the advanced glycation end product obtained from the measurement device and at least one of a plurality of other data of the subject that are different from the measurement value of the advanced glycation end product. The display device displays the advice information obtained from the advice device.

### Advantageous Effects of Invention

According to the present disclosure, advice information for the life of a subject is provided to the subject based on a measurement value of an advanced glycation end product of the subject, and in addition thereto, at least one of a plurality of other data of the subject that are different from the measurement value of the advanced glycation end product, and optimal advice can thus be provided to the subject for reviewing a lifestyle habit.

### BRIEF DESCRIPTION OF DRAWINGS

FIG. 1 is a diagram for illustrating an advice system according to an embodiment.

FIG. 2 shows an example of measurement values of skeletal muscle mass.

FIG. 3 shows an example of sleep data.

FIG. 4 shows an example of how a blood glucose level transitions as time elapses.

FIG. 5 shows an example of dietary log data.

FIG. 6 is a diagram for illustrating AGEs evaluation ranking.

FIG. 7 shows a configuration of an advice device according to an embodiment.

FIG. 8 is a diagram for illustrating a user identification information table that the advice device stores according to an embodiment.

FIG. 9 is a diagram for illustrating a viewing information table that the advice device stores according to an embodiment.

FIG. 10 shows an example of a relationship between AGEs and sleep for whether they are improved.

FIG. 11 shows an example of how an AGEs score is improved when advice information for a sleeping habit is provided.

FIG. 12 shows an example of how an AGEs score is improved when advice information for a sleeping habit and a dietary habit is provided.

FIG. 13 shows an example of a relationship between AGEs and skeletal muscle mass for whether they are improved.

FIG. 14 shows an example of prediction of skeletal muscle mass.

FIG. 15 shows an example of how an AGEs score is improved when advice information for an exercising habit is provided based on a predicted value of skeletal muscle mass.

FIG. 16 shows an example of how an AGEs score is improved when no advice information is provided.

FIG. 17 shows an example of how an AGEs score is improved when advice information for an exercising habit is provided based on a predicted value of skeletal muscle mass.

FIG. 18 shows an example of a display screen of a display device according to an embodiment.

FIG. 19 shows an example of a display screen of the display device according to an embodiment.

FIG. 20 shows an example of a display screen of the display device according to an embodiment.

FIG. 21 shows an example of a display screen of the display device according to an embodiment.

FIG. 22 shows an example of a display screen of the display device according to an embodiment.

FIG. 23 is a flowchart of an advice process performed by the advice device according to an embodiment.

### DESCRIPTION OF EMBODIMENTS

An embodiment will now be described in detail with reference to the drawings. Note that in the figure, identical or equivalent components are identically denoted and will not be described repeatedly in principle.

### [Configuration of Advice System]

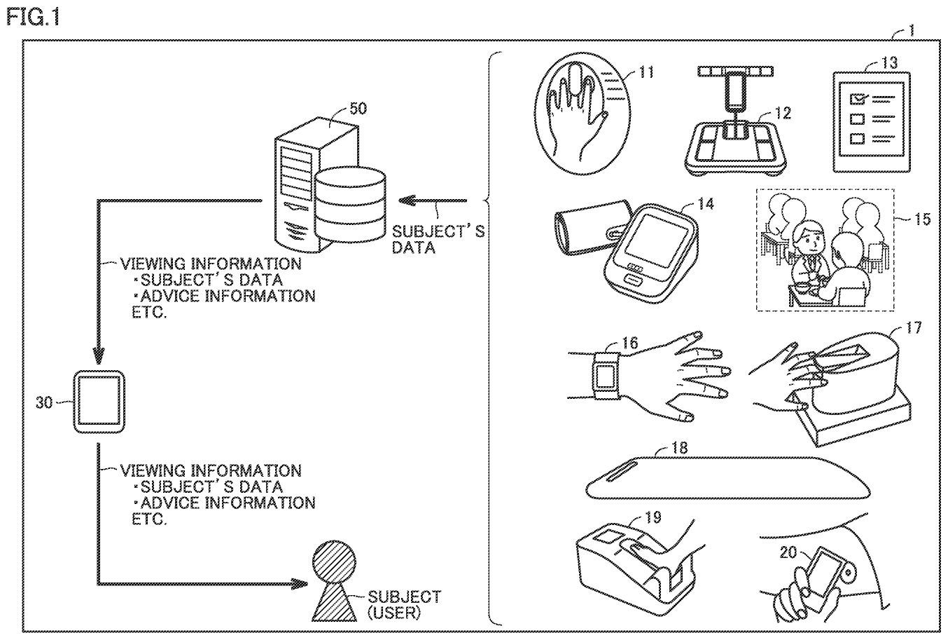

An advice system 1 according to an embodiment will now be described with reference to FIGS. 1 to 6. FIG. 1 shows an advice system 1 according to an embodiment. As shown in FIG. 1, advice system 1 comprises an AGEs measurement device 11 for measuring AGEs of a subject, a plurality of other measurement devices for measuring a plurality of other data of the subject that are different from a measurement value of AGEs, a display device 30, and an advice device 50. Note that the subject includes a healthy person, a person suspected of developing diabetes or a similar lifestyle-related disease or an age-related disease, a person already developing a lifestyle-related disease or an age-related disease, an elderly person using a care facility, and the like.

AGEs measurement device 11 non-invasively measures AGEs of the subject. Among a plurality of compounds contained in AGEs, there is a compound having a property of emitting fluorescence when irradiated with specific light. AGEs measurement device 11 measures the AGEs of the subject by utilizing a property of such a compound. Specifically, AGEs measurement device 11 receives with a light receiving element (not shown) fluorescence excited by light radiated to expose the skin of the subject thereto and measures the degree of accumulation of AGEs based on the intensity of the received fluorescence.

AGEs can change in one to several weeks, and accordingly, the subject measures AGEs at a frequency for example of one or several weeks. AGEs measurement device 11 obtains an AGEs measurement result, which is in turn transmitted to advice device 50 as the subject's data. The AGEs measurement result obtained by AGEs measurement device 11 include, for example, the intensity of the fluorescence received by AGEs measurement device 11 and a value obtained by converting the degree of accumulation of AGEs into a score. Note that the AGEs measurement result may include a corrected value obtained by correcting the values derived from converting both the intensity of the fluorescence received by AGEs measurement device 11 and the degree of accumulation of AGEs into a score.

The plurality of other measurement devices include a body composition meter 12, a sphygmomanometer 14, an activity meter 16, a vegetable intake measurement device 17, a sleep sensor 18, a bone densitometer 19, and a continuous blood glucose monitoring device 20.

Body composition meter 12 measures data for the body composition of the subject, such as skeletal muscle mass (hereinafter also referred to as “SMI”), body weight, body mass index (BMI), body fat percentage, visceral fat level, basal metabolic rate, and body age. For advice system 1 according to the embodiment, in particular, an example in which body composition meter 12 measures the SMI of the subject will be described.

In the diagnosis of sarcopenia, which is a condition in which a whole body's muscle mass decreases and muscle strength and physical function decrease, it is essential to measure a decrease in SMI as well as a decrease in walking speed and grip strength. While SMI declines with age or disease, SMI of the quadriceps, which is a muscle in the human body that develops through exercise, can be improved by improving lifestyle habits, such as diet and exercise. SMI is expressed by a value obtained by dividing the total limb skeletal muscle mass by the square of the height, with a reference value for sarcopenia defined for each gender.

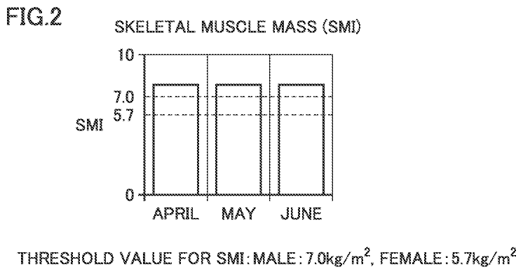

For example, FIG. 2 shows an example of measurement values of skeletal muscle mass (SMI). FIG. 2 is a graph having an axis of abscissas representing months in which measurement is done and an axis of ordinates representing SMI to show how SMI varies. As shown in FIG. 2, a reference value of 7.0 kg/m² is determined for males, and a reference value of 5.7 kg/m² is determined for females. When an SMI measured with body composition meter 12 is larger than or equal to these reference values, the subject does not have sarcopenia, whereas when the SMI measured with body composition meter 12 is less than these reference values, the subject is determined to have sarcopenia.

SMI is difficult to reflect in measurement values even if lifestyle habits are improved for several weeks, and it finally begins to reflect in measurement values after lifestyle habits are improved for several months. Therefore, the subject measures the SMI once every several months, for example. Body composition meter 12 obtains an SMI measurement result, which is in turn transmitted to advice device 50 as the subject's data, for example, at the timing of the measurement (at a frequency of once every several months).

Sphygmomanometer 14 measures the subject's blood pressure. Blood pressure can constantly change, and accordingly, the subject measures blood pressure, for example, every day at a fixed time. Sphygmomanometer 14 obtains a blood pressure measurement result, which is in turn transmitted to advice device 50 as the subject's data, for example, at the timing of the measurement (at a frequency of once per day).

Activity meter 16 measures data for an exercise of the subject, such as an exercise time, an exercise distance, a step count, a heart rate, and a calory consumed. The subject exercises while wearing activity meter 16 of a wearable type, and a measurement value can change depending on an amount of activity. Activity meter 16 obtains data for an exercise, which is in turn transmitted to advice device 50 as the subject's data, for example, at the timing of the measurement (at a frequency of once per day).

Vegetable intake measurement device 17 measures the subject's vegetable intake. Vegetable intake can constantly change, and accordingly, the subject measures vegetable intake, for example, every day at a fixed time. The vegetable intake measurement device 17 obtains a vegetable intake measurement result, which is in turn transmitted to advice device 50 as the subject's data, for example, at the timing of the measurement (at a frequency of once per day).

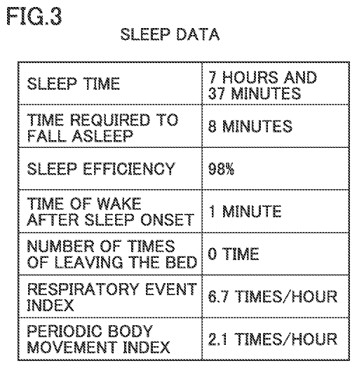

Sleep sensor 18 measures data for the subject's sleep. For example, FIG. 3 shows an example of sleep data. As shown in FIG. 3, sleep sensor 18 measures a sleep time, a time required to be asleep, sleep efficiency (a sleep time/a time of being in bed), a time of wake after sleep onset, the number of times of leaving the bed, a Respiratory Event Index (REI), a periodic body movement index, and the like. The subject sleeps daily using a comforter with sleep sensor 18 attached thereto, and a measurement value may change depending on how the subject sleeps. Sleep sensor 18 obtains data for sleep, which is in turn transmitted to advice device 50 as the subject's data, for example, at the timing of the measurement (at a frequency of once per day).

Bone densitometer 19 measures the subject's bone density. Bone density is difficult to reflect in measurement values even if lifestyle habits are improved for several weeks, and it finally begins to reflect in measurement values after lifestyle habits are improved for several months. Accordingly, the subject measures bone density once every two or three months, for example. Bone densitometer 19 obtains a bone density measurement result, which is in turn transmitted to advice device 50 as the subject's data, for example, at the timing of the measurement (at a frequency of once every several months).

Continuous glucose monitoring device 20 performs continuous glucose monitoring (CGM) to continuously measure the subject's blood glucose level over a determined period of time (for example of two weeks) by hypodermically puncturing a needle disposed at a sensor unit for measurement. Continuous glucose monitoring (CGM) is known to be effective as a method for confirming a disordered diet among other lifestyle habits, in particular. By continuous glucose monitoring, the subject can confirm whether a blood glucose level sharply rises and sharply falls, or spikes, and in that case, the subject can confirm how frequently it does so (or a frequency of blood glucose level spiking).

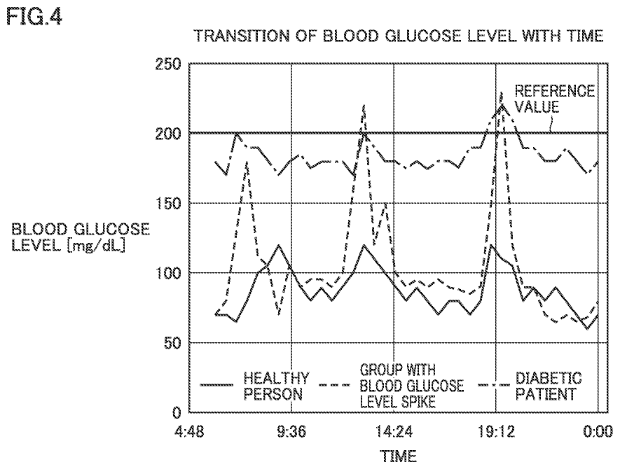

For example, FIG. 4 shows an example of how a blood glucose level transitions as time elapses. FIG. 4 is a graph having an axis of abscissas representing time and an axis of ordinates representing blood glucose level to show how a blood glucose level varies. In general, a postprandial blood glucose level of 140 mg/dL or less is normal, whereas a postprandial blood glucose level exceeding 200 mg/dL, which is a reference value, is said to be evidence for diagnosing diabetes. As shown in FIG. 4, when a blood glucose level spikes, it provides data showing that while a fasting blood glucose level is not different from that of a healthy person, a postprandial blood glucose level rapidly rises to the same value as that of a diabetic patient and exceeds 200 mg/dL, and subsequently rapidly drops and thus presents rapid variation.

The subject measures blood glucose level at a frequency of once per month, for example. Continuous glucose monitoring device 20 obtains a blood glucose level measurement result, which is in turn transmitted to advice device 50 as the subject's data, for example, at the timing of the measurement (at a frequency of once per month).

In addition to the measurement results obtained from AGEs measurement device 11, body composition meter 12, sphygmomanometer 14, activity meter 16, vegetable intake measurement device 17, sleep sensor 18, bone densitometer 19, and continuous glucose monitoring device 20 described above, advice device 50 obtains input data input by the subject or the supporter for the subject and the subject's dietary log data as the subject's data.

The input data input by the subject includes information of the subject reported by the subject or the supporter in response to a questionnaire 13, such as information on inflammation caused by a cold, an injury or the like, information on stress checking, information on smoking, and information on drinking. Note that the input data may be entered in a questionnaire sheet, or may be input using display device 30, as described hereinafter. Input data of inflammation etc. is transmitted to advice device 50 as the subject's data, for example, at the timing of the input.

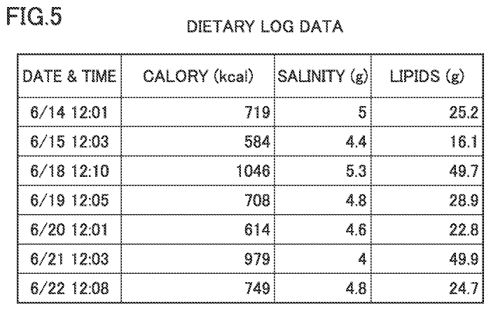

The dietary log data of the subject includes, for example, information such as calories or ingredients of meals provided from a dining hall 15 or the like. For example, FIG. 5 shows an example of dietary log data. As shown in FIG. 5, for example, for a dining hall of a company, a school or the like, calories, salinity, lipids, and the like are recorded as contents of meals provided at lunch time, and these log data are transmitted to advice device 50 as the subject's data.

As described above, advice device 50 obtains various data for confirming the subject's health condition measured with a plurality of measurement devices such as AGEs measurement device 11, body composition meter 12, sphygmomanometer 14, activity meter 16, vegetable intake measurement device 17, sleep sensor 18, bone densitometer 19, and continuous glucose monitoring device 20 as the subject's data, and also obtains input data input by the subject and the subject's dietary log data as the subject's data.

Display device 30 is owned or used by a user. Display device 30 is an information terminal capable of communicating with advice device 50 via a network, such as a desktop PC, a laptop PC, a smartphone, a smart watch, a wearable device, and a tablet PC. The user can use display device 30 to directly or indirectly access advice device 50 to obtain various types of information such as advice information stored in advice device 50, as will be described hereinafter.

The user is a user of a service provided by advice system 1 (hereinafter also referred to as an “information provision service”). Specifically, the user may be the subject or the supporter. Further, the user may be a family member, a relative, or a person related to the subject (e.g., an acquaintance) who has been authorized by the subject or the supporter to view a result of measurement on the subject.

The supporter is a person who supports the subject and includes staff of care facilities, life consultants of care facilities, hospital or clinic doctors, hospital or clinic nurses, fitness gym instructors or nutrition advisors.

Advice device 50 is managed by a service provider who provides an information provision service. The service provider may be a manufacturer of AGEs measurement device 11 that lends AGEs measurement device 11 to a user such as a subject or a supporter. Advice device 50 functions as a cloud computer to communicate with AGEs measurement device 11, body composition meter 12, sphygmomanometer 14, activity meter 16, vegetable intake measurement device 17, sleep sensor 18, bone densitometer 19, continuous glucose monitoring device 20, and display device 30.

Of the subject's data, an AGEs measurement value varies with lifestyle habits such as a dietary habit, an exercising habit, a sleeping habit, inflammation, and stress, and accordingly, a supporter who reviews the subject's lifestyle habits may provide advice to the subject to improve his/her lifestyle habits based on the AGEs measurement value.

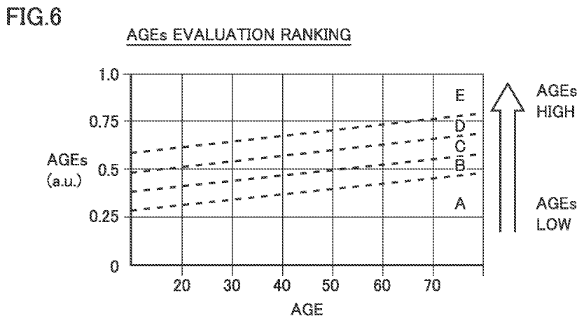

For example, FIG. 6 is a diagram for describing AGEs evaluation ranking. FIG. 6 indicates a subject's age along the axis of abscissas and AGEs score along the axis of ordinates to show a criterion for evaluation for AGEs. Note that an AGEs score is a value obtained by converting an AGEs measurement value obtained by AGEs measurement device 11 into a score between 0 and 1.0.

As shown in FIG. 6, a plurality of reference values are provided stepwise for AGEs scores, and based on comparing an AGEs score with the plurality of reference values, an AGEs measurement value can be ranked in a five-step evaluation of A to E. For example, a smaller AGEs measurement value has an AGEs evaluation rank closer to “A” and an AGEs measurement value with an AGEs evaluation rank of “A” has the best evaluation, whereas a larger AGEs measurement value has an AGEs evaluation rank closer to “E” and an AGEs measurement value with an AGEs evaluation rank of “E” has the worst evaluation.

The reference values shown in FIG. 6 for AGEs evaluation ranking are one example, and any value may be set as a reference value. Further, the reference values for AGEs evaluation ranking may not be limited by the age of the subject and may differ based on gender.

The supporter can use an AGEs measurement value of the subject obtained through AGEs measurement device 11 and such reference values as shown in FIG. 6 to evaluate the AGEs measurement value of the subject and accordingly provide advice to the subject to improve a lifestyle habit.

However, an AGEs measurement value varies with various lifestyle habits, such as a dietary habit, an exercising habit, a sleeping habit, inflammation, and stress, and when attempting to provide advice based on the AGEs measurement value alone, the supporter may not be able to specifically determine what lifestyle habit should be improved, and the supporter may not be able to provide optimal advice to the subject.

For example, it is known that workers of the working age generation have exercising habits less than elderly people, and when the subject is a worker of the working age generation, the supporter often advises the subject to be first aware of establishing a regular exercising habit. Thereafter, in order to present advice that the subject would accept, the supporter takes time to counsel with the subject, while searching for a lifestyle habit that prevents the subject's health condition from being improved. Such a work depends on a specific individual, and a supporter who is poor at counseling may not be able to provide appropriate advice to the subject. Further, even a supporter who is good at counseling also requires a long period of time before providing appropriate advice.

On the other hand, if the supporter provides advice comprehensively across generally known lifestyle habits, there is a possibility that the supporter may unintentionally deny a lifestyle habit that the subject is already working on improving it. This may reduce the subject's positive motivation to improve the lifestyle habit and the subject may significantly be unwilling to accept the advice.

Accordingly, in advice system 1 according to an embodiment, advice device 50 is not simply configured to generate advice information for the life of the subject based on an AGEs measurement value obtained by AGEs measurement device 11 alone; rather, it is also configured to do so based on at least one of a plurality of other data of the subject that are different from the AGEs measurement value.

Specifically, once advice device 50 obtains the subject's data, the advice device stores the subject's obtained data together with the subject's data obtained previously. Advice device 50 generates advice information for the life of the subject based on an AGEs measurement value included in the subject's data and at least one of the plurality of other data of the subject other that are different from the AGEs measurement value included in the subject's data. Advice device 50 also generates advice information based on at least one of the other data of the subject obtained on a date correlated with a date on which the AGEs measurement was obtained, e.g., a date closest to the date on which the AGEs measurement was obtained.

Advice device 50 stores the subject's data and the advice information generated based on the subject's data as viewing information viewable by a user such as the subject, the supporter, and a viewer. The advice information includes advice for at least one of a dietary habit, an exercising habit, a sleeping habit, and a mental health condition of the subject.

When the user requests the viewing information using display device 30, advice device 50 outputs the viewing information to display device 30 in response to the request received from display device 30. Display device 30 displays the viewing information obtained from advice device 50.

Advice device 50 can thus generate advice information based on an AGEs measurement value and in addition thereto a plurality of other data of the subject that are different from the AGEs measurement value, and provide the advice information to a user such as the subject or a supporter. In addition, advice device 50 generating the advice information based on data selected from the plurality of other data of the subject can provide the user with optimal advice for reviewing a lifestyle habit. Further, the advice device allows even a supporter who is poor at counseling to provide persuasive advice to the subject, and can thus avoid the subject's unwillingness to accept the advice. Further, the advice device also allows a supporter who is good at counseling to provide appropriate advice in a relatively short period of time.

### [Configuration of Advice Device]

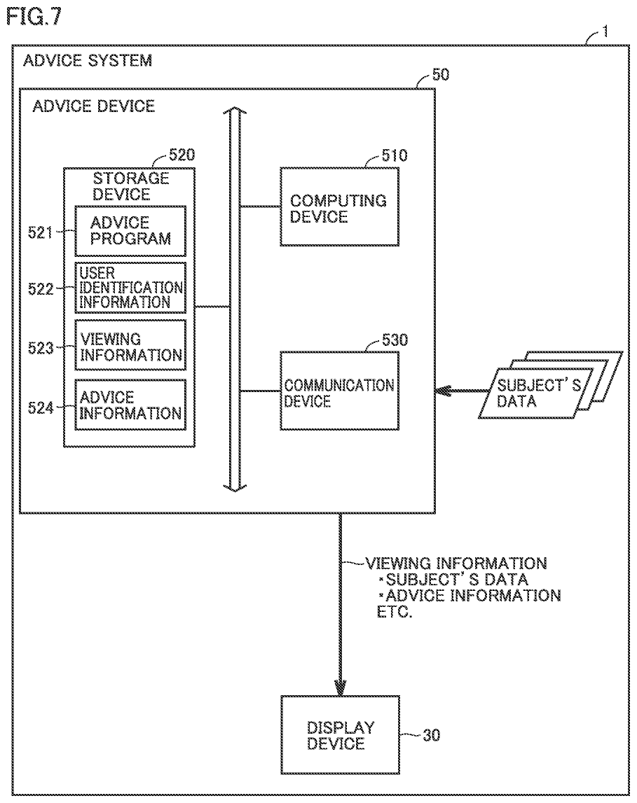

A configuration of advice device 50 will now be described with reference to FIGS. 7 to 9. FIG. 7 shows a configuration of advice device 50 according to an embodiment. As illustrated in FIG. 7, advice device 50 comprises a computing device 510, a storage device 520, and a communication device 530.

Computing device 510 is a computer (a computing entity) that executes various processes in accordance with various programs. Computing device 510 is configured by a computer such as a processor. The processor includes at least one of a CPU (Central Processing Unit), an FPGA (Field Programmable Gate Array), a GPU (Graphics Processing Unit), and an MPU (Multi Processing Unit), for example. Furthermore, computing device 510 may comprise a storage unit for storing program code, working memory, or the like when the processor executes various programs. The storage unit may be one or more non-transitory computer readable media. The storage unit may include volatile memory such as DRAM (Dynamic Random Access Memory) and SRAM (Static Random Access Memory), as well as nonvolatile memory such as ROM (Read Only Memory) and flash memory. Note that computing device 510 (or the processor) is not limited to a processor in a narrow sense that executes processing in a stored program method, such as a CPU or an MPU, and may include a hardwired circuit such as an ASIC, a GPU, or an FPGA. The processor may be configured with processing circuitry in which processing is defined in advance by computer readable code and/or a hardwired circuit. Note that the processor may be configured by a single chip or may be configured by a plurality of chips. Further, the processor and associated processing circuitry may be composed of a plurality of computers interconnected in a wired or wireless manner via a local area network, a wireless network, or the like. The processor and associated processing circuitry may be configured by a cloud computer that remotely performs an operation based on input data and outputs a result of the operation to another remotely located device.

Storage device 520 is one or more computer readable storage media and includes a nonvolatile memory, such as an HDD (Hard Disk Drive) and an SSD (Solid State Drive). Storage device 520 stores various programs and data such as an advice program 521 executed by computing device 510, user identification information 522 referred to by computing device 510, viewing information 523 viewable by a user via display device 30, and advice information 524 prepared in advance.

Note that storage device 520 may include a volatile memory, such as DRAM and SRAM, and a non-volatile memory, such as ROM and flash memory. Further, computing device 510 may comprise a media reading device (not shown). Computing device 510 may accept a removable disk, which is one or more computer readable storage media, through the media reading device and may retrieve various programs and data, such as advice program 521, user identification information 522 and advice information 524 from the removable disk.

Advice program 521 defines various types of instructions to be executed to generate advice information based on the subject's data obtained via communication device 530.

User identification information 522 includes information about the user, such as a user ID, a password, and a user name. Advice device 50 can identify the user using user identification information 522.

Viewing information 523 includes: subject information including information on the subject; AGEs information including information on an AGEs measurement value of the subject obtained from AGEs measurement device 11; SMI information including information on an SMI of the subject obtained from body composition meter 12; inflammation information including information on inflammation of the subject input by the user; blood pressure information including information on blood pressure of the subject obtained from sphygmomanometer 14; dietary information including dietary log data of the subject; exercise information including data on an exercise done by the subject obtained from activity meter 16; vegetable intake information including information on vegetable intake of the subject obtained from vegetable intake measurement device 17; sleep information including information on sleep of the subject obtained from sleep sensor 18; bone density information including information on bone density of the subject obtained from bone densitometer 19; and blood glucose level information including information on a blood glucose level of the subject obtained from continuous glucose monitoring device 20; and advice information generated based on the subject's data.

Advice information 524 includes a plurality of types of advice for the life of the subject, and is stored in storage device 520 so as to be selectable depending on the content of the subject's data. Computing device 510 selects at least one piece of advice from the plurality of types of advice included in advice information 524 based on the subject's data, and includes the selected advice in viewing information 523. The advice for the life of the subject includes, for example, advice for the subject's dietary habit, exercising habit, sleeping habit, mental health condition, etc.

Communication device 530 receives the subject's data from AGEs measurement device 11, body composition meter 12, sphygmomanometer 14, activity meter 16, vegetable intake measurement device 17, sleep sensor 18, bone densitometer 19, and continuous glucose monitoring device 20 through wired or wireless communication. Further, communication device 530 receives input data, such as information on inflammation, input by the user, and dietary log data. Further, communication device 530 transmits the viewing information to the display device through wired or wireless communication.

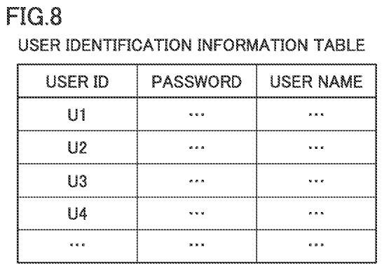

FIG. 8 is a diagram for describing a user identification information table stored by advice device 50 according to an embodiment. Advice device 50 stores user identification information 522 using the user identification information table shown in FIG. 8.

As shown in FIG. 8, the user identification information table stores a variety of types of information for a user, such as a user ID, a password, and a user name, as user identification information 522. Each user utilizing the information provision service is identified by user identification information 522. For example, a first user is assigned “U1” as the user ID, and a second user is assigned “U2” as the user ID.

The user ID, the password, and the user name, among user identification information 522, are entered by each user through display device 30. Display device 30 outputs the input user identification information 522 to advice device 50. Advice device 50 stores user identification information 522 obtained from display device 30 in storage device 520 by storing it in the user identification information table.

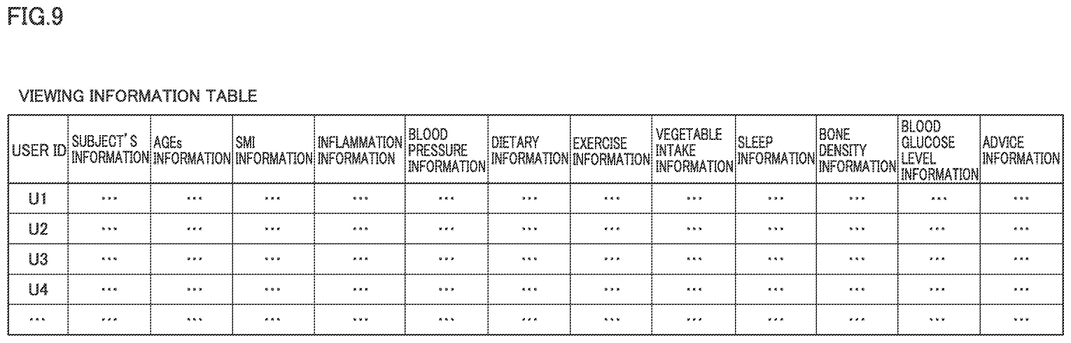

FIG. 9 is a diagram for describing the viewing information table stored by advice device 50 according to an embodiment. Advice device 50 stores viewing information 523 using the viewing information table shown in FIG. 9.

As shown in FIG. 9, the viewing information table stores various types of information viewable by the user, such as subject information, AGEs information, SMI information, inflammation information, blood pressure information, dietary information, exercise information, vegetable intake information, sleep information, bone density information, blood glucose level information, and advice information, in association with a user ID.

Advice device 50 evaluates an AGEs measurement value, an SMI, information on inflammation, a blood pressure, dietary log data, data on exercise, an amount of vegetable intake, information on sleep, a bone density, and a blood glucose level included in the subject's data obtained, and ranks the subject's each data to correspond to a result of the evaluation. For example, advice device 50 ranks the subject's each data by five-rank evaluation of A to E as shown in FIG. 6. Advice device 50 stores the subject's each data and a rank therefor in the viewing information table (or viewing information 523). Note that the number of ranking stages for each data of the subject may vary depending on the data.

### [One Example of Advice]

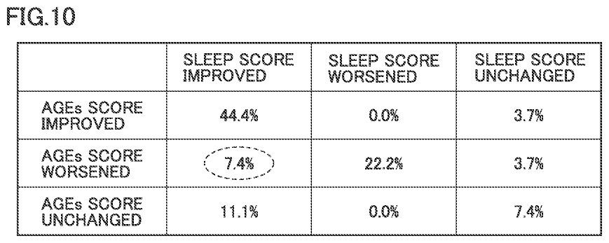

An example of advice for a subject in advice system 1 will now be described with reference to FIGS. 10 to 18. FIG. 10 shows an example of a relationship between AGEs and sleep for whether they are improved.

As shown in FIG. 10, of the total, 44.4% of people have improved AGEs scores and improved sleep scores. Of the total, 22.2% of people have worsened AGEs scores and worsened sleep scores. Of the total, 7.4% of people have no change in their AGEs and sleep scores. The sum of these percentages is 74%, indicating that there is a correlation between AGEs scores and sleep scores. That is, it can be seen that advice to improve a sleeping habit is important to improve an AGEs score.

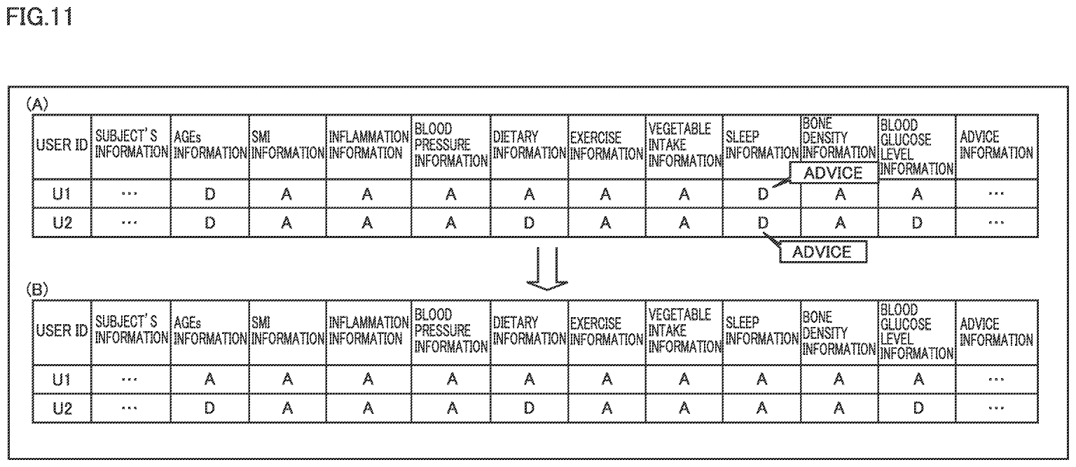

For example, FIG. 11 shows an example of how an AGEs score is improved when advice information is provided for a sleeping habit. As shown in FIG. 11(A), for the user having the user ID “U1”, an evaluation result of rank D is stored as the subject's AGEs information, an evaluation result of rank D is stored as the subject's sleep information, and an evaluation result of rank A is stored for the subject's other data.

As the AGEs evaluation is rank D, which is a result worse than rank A indicating a health condition that can be said to be normal, and accordingly, in order to recover the AGEs evaluation to rank A, advice device 50 generates advice information for improving a lifestyle habit. In doing so, advice device 50 generates advice information for improving the sleeping habit as a lifestyle habit, in particular, and provides the advice information to the subject because, of the plurality of other data of the subject that are different from AGEs, the sleep data is evaluated as rank D, which is a result worse than rank A indicating a health condition that can be said to be normal.

The subject improves the sleeping habit in accordance with the advice information provided from advice device 50, and the evaluation for the sleep is changed from rank D to rank A as shown in FIG. 11(B). As the sleeping habit is improved, the AGEs evaluation is changed from rank D to rank A.

Returning to FIG. 10, it can be noted that, of the total, 7.4% of people have worsened AGEs scores despite improved sleep scores.

For example, as shown in FIG. 11(A), for the user having the user ID “U2”, an evaluation result of rank D is stored as the subject's AGEs information, an evaluation result of rank D is stored as the subject's sleep information, an evaluation result of rank D is stored as the subject's dietary information, and an evaluation result of rank D is stored as the subject's blood glucose level information.

As well as for the user having the user ID “U1”, when advice device 50 generates advice information for improving a sleeping habit alone and provides the advice information to the subject, the subject has an evaluation for sleep changed from rank D to rank A as shown in FIG. 11(B). However, the AGEs evaluation does not change from rank D to rank A when the sleeping habit is alone improved. That is, for this subject, not only the sleeping habit but also a dietary habit to which a diet and a blood glucose level relates are causes of the poor AGEs evaluation.

Accordingly, advice device 50 according to an embodiment generates advice information for the dietary habit in addition to the sleeping habit, and provides the advice information to the subject.

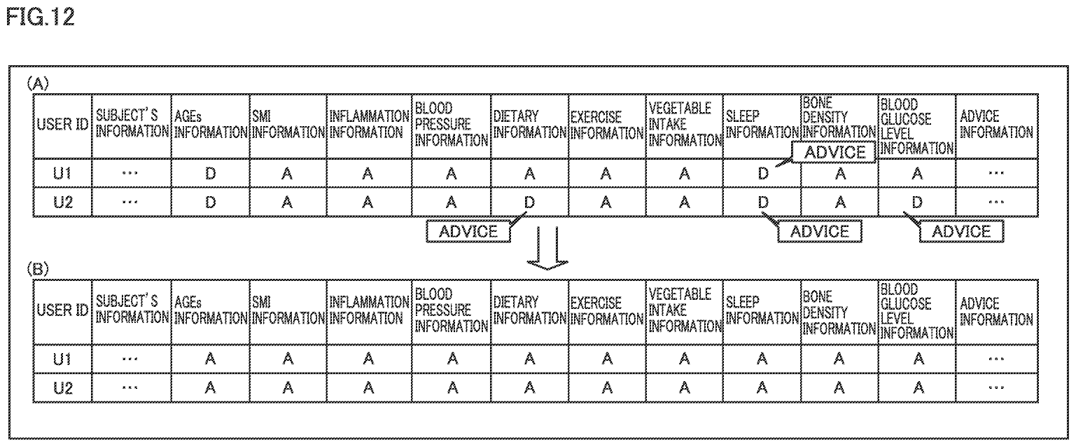

For example, FIG. 12 shows an example of how an AGEs score is improved when advice information is provided for a sleeping habit and a dietary habit. As shown in FIG. 12(A), the user having the user ID “U2” is evaluated as rank D for sleep data among the plurality of other data of the subject that are different from AGEs, and accordingly, advice device 50 generates advice information for improving a sleeping habit as a lifestyle habit and provides the advice information to the subject, and furthermore, as the user is also evaluated as rank D for diet and blood glucose level among the plurality of other data of the subject that are different from AGEs, the advice device generates advice information for improving a dietary habit as a lifestyle habit and provides the advice information to the subject.

The advice for improving the dietary habit includes practicing vegetable first to eat vegetables first, not overeating carbohydrates such as rice and ramen, and not eating fast as quickly as 5 minutes, etc.

When the subject improves the sleeping habit and the dietary habit in accordance with the advice information provided from advice device 50, then, as shown in FIG. 12(B), the evaluation for sleep is changed from rank D to rank A, the evaluation for diet is changed from rank D to rank A, and the evaluation for blood glucose level is changed from rank D to rank A. As the sleeping habit and the dietary habit are improved, the AGEs evaluation is changed from rank D to rank A.

As described above, advice device 50 generates advice information while considering not only one piece of a subject's data such as sleep but also a plurality of data of the subject obtained by adding diet and blood glucose level to sleep based on each data of the subject, and the advice device can provide optimal advice to the subject for reviewing lifestyle habits.

An AGEs score may rapidly change when an inflammatory response is caused by catching a cold or the like. Although it is imaginable that an inflammatory response per se has an aspect of promoting production of AGEs, a person with an inflammatory response has a diet easily causing a large intake of isomerized glucose syrup and is often unable to exercise to promote metabolism. Such a state continuing for several days also invites a gradually worsening AGEs score. Furthermore, as the inflammatory response prolongs, the AGEs score also remains worsened. That is, when inflammation is caused, an inflammation score is initially worsened and subsequently an inflammatory response causes worsened dietary and exercise scores and hence a worsened AGEs score.

When advice device 50 refers to the subject's data, the advice device may refer to not only inflammation of worse evaluation but also dietary information, exercise information, blood glucose level information, and the like, and if the information has a poor evaluation, the advice device may generate advice information for the dietary habit and the exercising habit and provide the advice information to the subject.

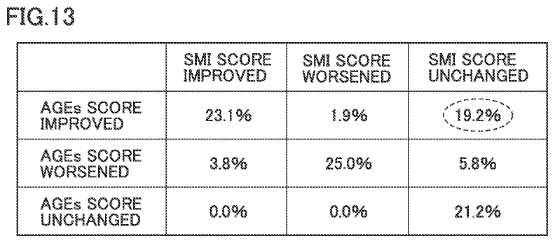

FIG. 13 shows an example of a relationship between AGEs and skeletal muscle mass for whether they are improved. As shown in FIG. 13, of the total, 23.1% of people have improved AGEs scores and improved SMI scores. Of the total, 25.0% of people have worsened AGEs scores and worsened SMI scores. Of the total, 21.2% of people have no change in their AGEs and SMI scores. The sum of these percentages is 69.3%, indicating that there is a correlation between AGEs scores and SMI scores. That is, in order to improve an AGEs score, it can be seen that advice for improving an exercising habit for SMI is important.

Accordingly, when a subject has the plurality of other data that are different from AGEs with an SMI evaluated to have a result worse than rank A, advice device 50 generates advice information for improving an exercising habit as a lifestyle habit and provides the advice information to the subject.

Herein, it can be noted that, of the total, 19.2% of people have improved AGEs scores despite unchanged SMI scores. A reason for this is considered as follows: while AGEs change within one to a few weeks in general, SMI hardly changes as quickly as AGEs within a few weeks, and only begins to reflect improvement in a measurement value after several months of lifestyle changes.

Therefore, when advice device 50 generates advice information based on the currently obtained SMI, the advice device may be unable to provide optimal advice to the subject. For example, while advice device 50 provides advice based on an SMI evaluation of rank D to improve an exercising habit, the subject may have already been working on improving the exercising habit and still have the improvement unreflected in SMI.

Accordingly, advice device 50 predicts an SMI based on an AGEs measurement value and generates advice information based on a resultant, predicted SMI. Specifically, advice device 50 predicts an SMI based on a change in time series of a previously obtained and stored AGEs measurement value, and a previously obtained and stored SMI measurement value, and stores a value predicted for the SMI (hereinafter also referred to as an “SMI prediction value”). When a poorly evaluated SMI and a satisfactorily evaluated SMI prediction value are obtained, advice device 50 provides the subject with advice information indicating an improved exercising habit.

Regardless of whether the subject is male or female, the smaller the AGEs measurement value, the larger the SMI measurement value, and the larger the AGEs measurement value, the smaller the SMI measurement value. Thus, there is a correlation between AGEs and SMI, and advice device 50 uses such a correlation between AGEs and SMI to predict an SMI for some timing in the future based on a change in time series of an AGEs measurement value, and an SMI measurement value.

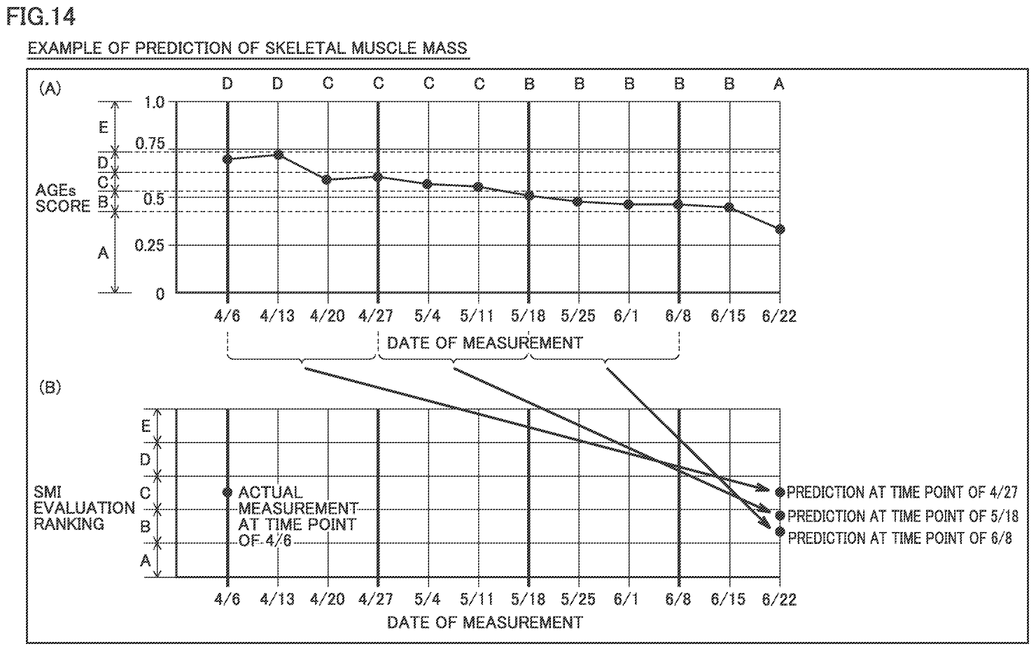

FIG. 14 shows an example of prediction of skeletal muscle mass. With reference to FIG. 14 will be described an example in which advice device 50 calculates an SMI prediction value using a subject's AGEs measurement values obtained every week for 4/6 to 6/22. FIG. 14(A) is a graph having an axis of abscissas representing date and an axis of ordinates representing AGEs measurement scoring to show how the subject's AGEs measurement value changes in time-series. FIG. 10(B) is a graph having an axis of abscissas representing date and an axis of ordinates representing SMI evaluation ranking to show how the subject's SMI evaluation rank changes in time-series.

As noted above, AGEs can change generally in one to a few weeks in response to an improved lifestyle habit. Therefore, the subject typically, periodically measures AGEs every few weeks using AGEs measurement device 11. For example, the subject uses AGEs measurement device 11 to measure AGEs every week for 4/6 to 6/22. Advice device 50 obtains AGEs measurement values from AGEs measurement device 11 every week for 4/6 to 6/22.

In contrast to AGEs, SMI hardly changes in a few weeks, and begins to be reflected in a measurement value after a lifestyle habit is improved for a few months. Accordingly, the subject typically measures SMI every few months using body composition meter 12. For example, when the subject measures an SMI on 4/6 using body composition meter 12, the subject is scheduled to measure an SMI a few months later, or on 6/22. Advice device 50 obtains an SMI measurement value at a time point of 4/6 from AGEs measurement device 11.

After obtaining the SMI measurement value at the time point of 4/6, advice device 50 calculates at a subsequent timed prediction time point an SMI for the subject for the next SIM measurement date, i.e., 6/22, as an SMI prediction value. Further, advice device 50 calculates the SMI prediction value based on a change in time series of the AGEs measurement value for a predetermined period of time retroactively from the prediction time point.

For example, when calculating an SMI prediction value for 6/22 at a time point of 4/27, advice device 50 reads AGEs measurement values obtained over the past one week retroactively from 4/27, i.e., on 4/6, 4/13, 4/20, and 4/27, from storage device 520. Advice device 50 calculates an SMI prediction value for 6/22 based on a change in time series of the AGEs measurement value for every week between 4/6 and 4/27, as read, and the SMI measurement value obtained previously on 4/6. Then, advice device 50 compares the calculated SMI prediction value with a reference value to calculate an SMI evaluation rank.

When calculating an SMI prediction value for 6/22 at a time point of 5/18 advice device 50 reads AGEs measurement values obtained over the past one week retroactively from 5/18, i.e., on 4/27, 5/4, 5/11, and 5/18, from storage device 520. Advice device 50 calculates an SMI prediction value for 6/22 based on a change in time series of the AGEs measurement value for every week between 4/27 and 5/18, as read, and the SMI measurement value obtained previously on 4/6. Then, advice device 50 compares the calculated SMI prediction value with a reference value to calculate an SMI evaluation rank.

When calculating an SMI prediction value for 6/22 at a time point of 6/8 advice device 50 reads AGEs measurement values obtained over the past one week retroactively from 6/8, i.e., on 5/18, 5/25, 6/1, and 6/8, from storage device 520. Advice device 50 calculates an SMI prediction value for 6/22 based on a change in time series of the AGEs measurement value for every week between 5/18 to 6/8, as read, and the SMI measurement value obtained previously on 4/6. Then, advice device 50 compares the calculated SMI prediction value with a reference value to calculate an SMI evaluation rank.

In the example shown in FIG. 14, the AGEs measurement values obtained from 4/6 to 6/8 are gradually getting smaller and thus improving, and accordingly, advice device 50 predicts at each prediction time point that the SMI evaluation rank for 6/22 will also gradually improve. The thus obtained SMI prediction value and SMI evaluation rank are stored in the viewing information table of FIG. 9 as SMI information.

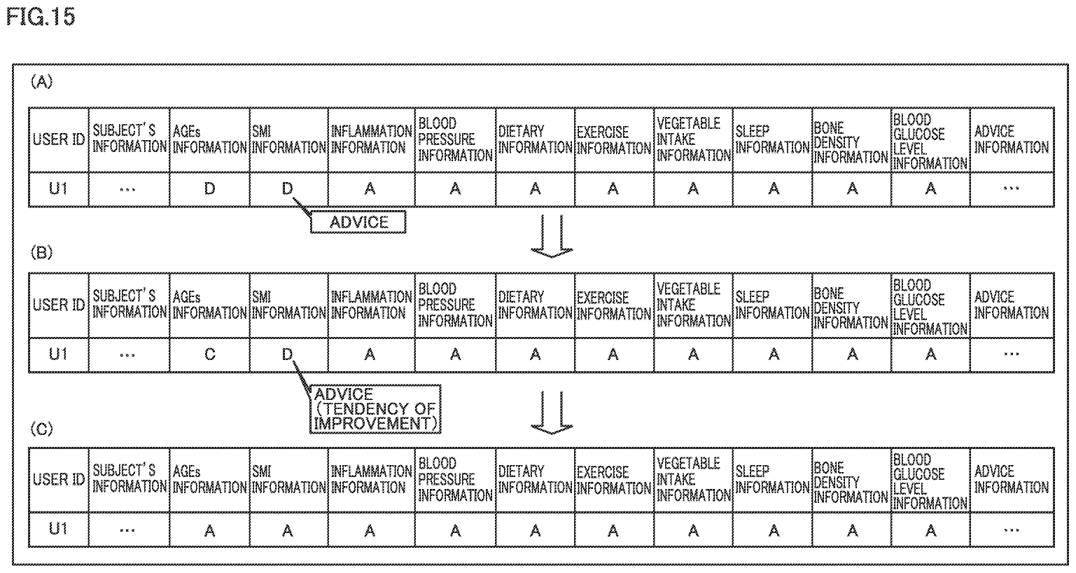

FIG. 15 shows an example of how an AGEs score is improved when advice information for an exercising habit is provided based on a predicted value of skeletal muscle mass.

As shown in FIG. 15(A), for the user having the user ID “U1”, an evaluation result of rank D is stored as the subject's AGEs information, and an evaluation result of rank D is stored as the subject's SMI information. Advice device 50 predicts an SMI based on an AGEs measurement value, and provides advice for improving an exercising habit to the subject as advice information based on the SMI prediction value.

When the subject improves the exercising habit in accordance with the advice information provided from advice device 50, the AGEs evaluation changes from rank D to rank C, as shown in FIG. 15(B). In contrast, the SMI evaluation remains at rank D as the lifestyle habit is reflected in a measurement value more slowly for SMI than AGEs.

However, advice device 50 predicts an SMI based on an AGEs measurement value and as a result if the SMI prediction value is large and improved, the advice device provides the subject with, as advice information based on the SMI prediction value, advice for example recognizing that the subject has improved the exercising habit and indicating that the subject's SMI tends to improve.

When the subject continues to improve the exercising habit in accordance with the advice information provided from advice device 50, then, as shown in FIG. 15(C), the AGEs evaluation is changed from rank C to rank A, and the SMI evaluation is also changed from rank D to rank A.

Thus, when an evaluation of an AGEs measurement value goes up and an evaluation of an SMI does not change, advice device 50 predicts an SMI based on an obtained SMI measurement value and a change in time series of the AGEs measurement value, and the advice device generates advice information based on the obtained SMI prediction value.

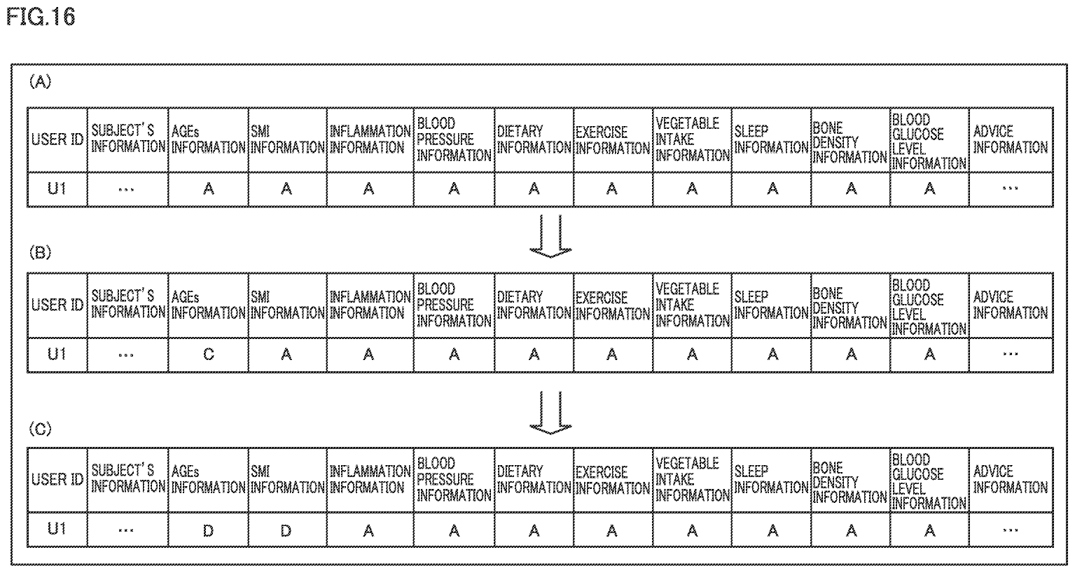

FIG. 16 shows an example of how an AGEs score is improved when no advice information is provided. As shown in FIG. 16(A), for the user having the user ID “U1”, an evaluation result of rank A is stored as the subject's AGEs information, and an evaluation result of rank A is also stored for the subject's other data.

Thereafter, as shown in FIG. 16(B), the AGEs evaluation is changed from rank A to rank C for the sake of illustration. While the AGEs evaluation has worsened because of lack of exercise, the subject has an SMI evaluation remaining at rank A because a lifestyle habit is reflected in a measurement value more slowly for SMI than AGEs. In such a case, advice device 50 will not provide any advice information to the subject as the subject's data other than AGEs is evaluated as rank A. In that case, it is expected that the subject does not improve his/her lifestyle habits, and as shown in FIG. 16(C), the AGEs evaluation is further worsened and changed from rank C rank to rank D, and furthermore, the SMI evaluation is also changed from rank A to rank D.

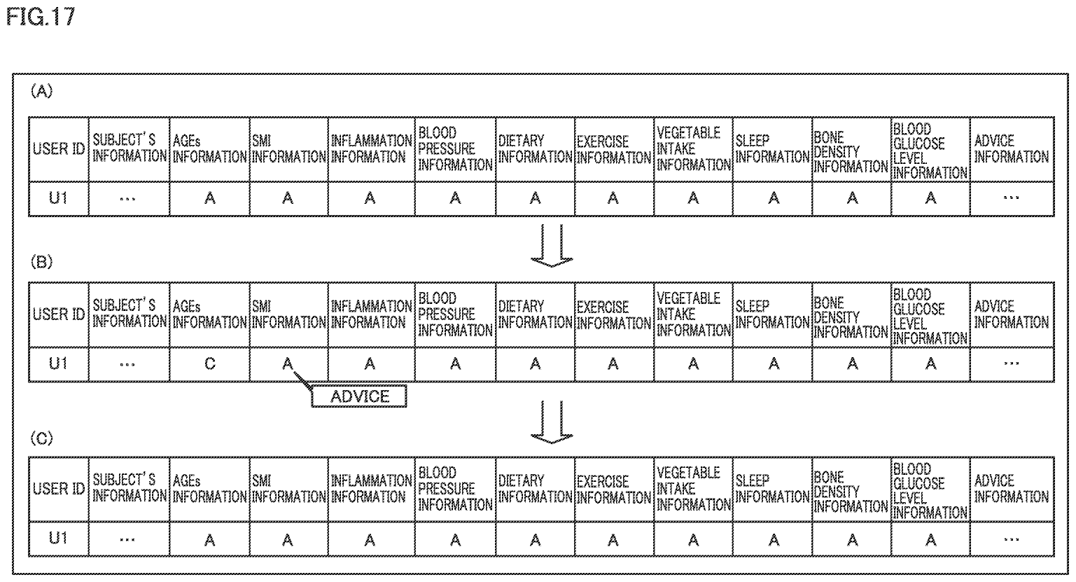

In contrast, FIG. 17 shows an example of how an AGEs score is improved when advice information for an exercising habit is provided based on a predicted value of skeletal muscle mass. As shown in FIG. 17(A), for the user having the user ID “U1”, an evaluation result of rank A is stored as the subject's AGEs information, and an evaluation result of rank A is also stored for the subject's other data.

Thereafter, as shown in FIG. 17(B), the AGEs evaluation is changed from rank A to rank C for the sake of illustration. In such a case, advice device 50 predicts an SMI based on an AGEs measurement value and as a result if the SMI prediction value is small and thus worsened, advice device 50 provides advice for improving an exercising habit to the subject as advice information based on the SMI prediction value.

When the subject improves the exercising habit in accordance with the advice information provided from advice device 50, the AGEs evaluation changes from rank C to rank A as shown in FIG. 17(C).

Thus, when an evaluation of an AGEs measurement value goes down and an evaluation of an SMI does not change, advice device 50 predicts an SMI based on an obtained SMI measurement value and a change in time series of the AGEs measurement value, and the advice device generates advice information based on the obtained SMI prediction value.

Note that once advice device 50 provides advice information to a subject and the subject leads a life following the advice information and thereafter when the subject achieves an AGEs measurement value with an evaluation going up, the advice device may generate new advice to include continuing the life following the advice information and provide the new advice information to the subject via display device 30.

On the other hand, once advice device 50 provides advice information to a subject and the subject leas a life following the advice information and thereafter when the subject still does not achieve an AGEs measurement value with an evaluation going up, the advice device may notify the subject of the once provided advice information once again via display device 30.

### [Example of Displaying Viewing Information]

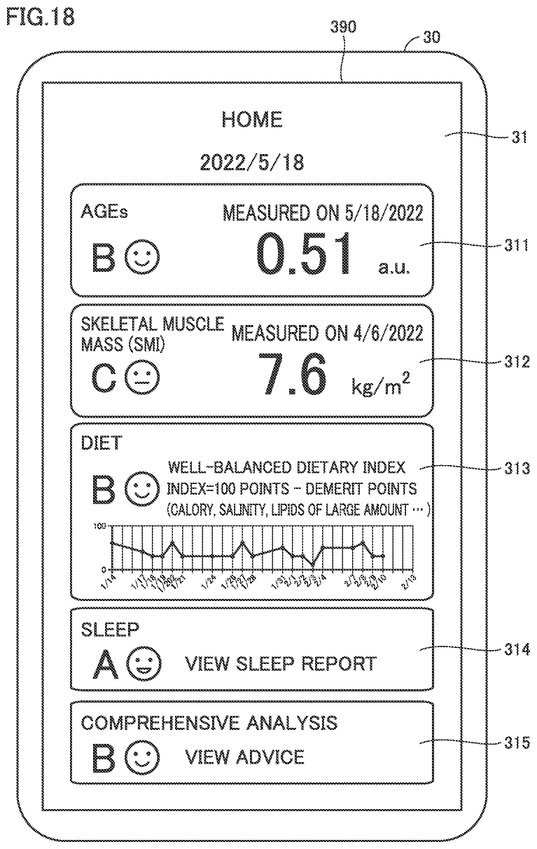

Referring to FIG. 18 to FIG. 22, an example of displaying the viewing information will be described. FIGS. 18 to 22 show examples of display screens of display device 30 according to an embodiment.

When the user executes an application program using display device 30 for using the information provision service, display device 30 displays a login screen (not shown) on a display 390. When the user enters a user ID and a password on the login screen, display device 30 outputs the user ID and the password to advice device 50. When advice device 50 authenticates the user based on the user ID and the password, then, as shown in FIG. 18, display device 30 displays a home screen 31 on display 390.

Home screen 31 includes an image 311 for viewing AGEs information, an image 312 for viewing SMI information, an image 313 for viewing dietary information, an image 314 for viewing sleep information, and an image 315 for viewing advice information. Home screen 31 may include an image for viewing other information such as blood pressure information and blood glucose level information.

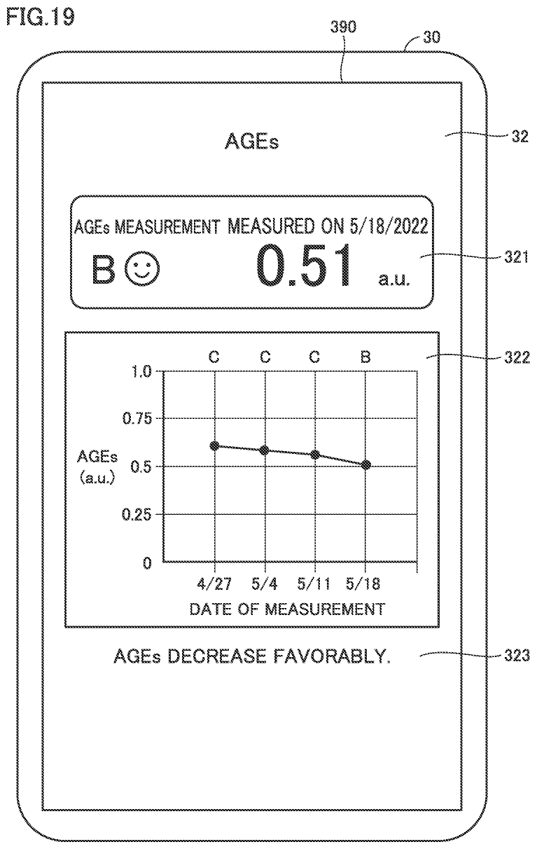

Image 311 for example indicates an AGEs score corresponding to the most recently measured AGEs measurement value, and a result of ranking that AGEs score. In the FIG. 18 example, image 311 indicates “0.51” as an AGEs score measured on May 18, 2022, and indicates “B” as a rank for the AGEs. When the user selects (for example touches) image 311, then, as shown in FIG. 19, display device 30 displays an AGEs viewing screen 32 on display 390 for viewing the AGEs information.

AGEs viewing screen 32 includes an image 321 indicating the most recently measured AGEs measurement value, an image 322 indicating in time series how the AGEs measurement value changed in the past, and an image 323 indicating comments on the AGEs measurement value. Thus, the user can view the AGEs information of the subject using display device 30.

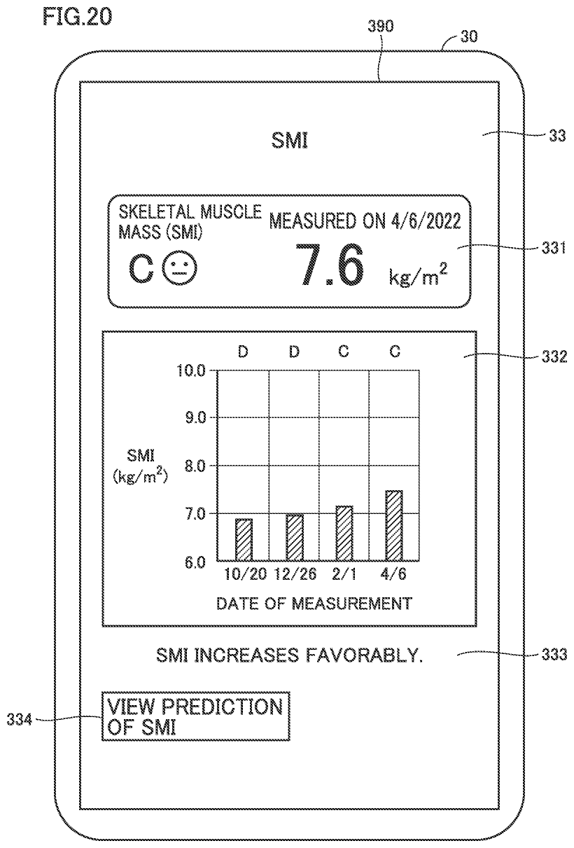

Returning to FIG. 18, image 312 indicates, for example, the most recently measured SMI measurement value and a result of ranking the SMI measurement value. In the example of FIG. 18, image 312 indicates “7.6” as an SMI measurement value measured on Apr. 6, 2022, and indicates “C” as a rank for the SMI. When the user selects image 312, then, as illustrated in FIG. 20, display device 30 displays an SMI viewing screen 33 on display 390 for viewing the SMI information.

SMI viewing screen 33 includes an image 331 indicating the most recently measured SMI measurement value, an image 332 indicating in time series how the SMI measurement value changed in the past (for example in the past six months), and an image 333 indicating comments on the SMI measurement value. Thus, the user can view the SMI information of the subject using display device 30.

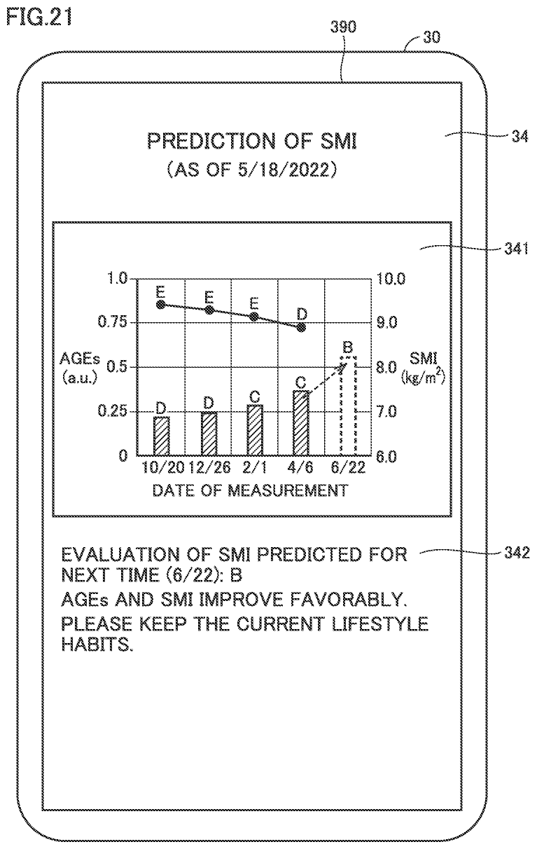

Further, SMI viewing screen 33 includes an image 334 for viewing an SMI prediction value. When the user selects image 334, then, as illustrated in FIG. 21, display device 30 displays an SMI prediction screen 34 on display 390 for viewing the SMI prediction value.

SMI prediction screen 34 includes an image 341 indicating an SMI prediction value for the next SMI measurement date (6/22 in this example) as calculated at the current time point (as of 5/18 in this example) and an image 342 indicating advice based on the SMI prediction value for the subject's lifestyle habits. In this example, as a result of predicting an SMI at a time point of 5/18, an image 351 indicates an SMI evaluation rank improved from “C” to “B” for 6/22. Further, image 342 indicates comments indicating as advice for lifestyle habits that AGEs and SMI are improving favorably. Thus, the user can view an SMI prediction value for the subject, an SMI evaluation rank corresponding to the SMI prediction value, advice based on the SMI prediction for lifestyle habits, and the like using display device 30.

Returning to FIG. 18, image 313 shows, for example, dietary log data for the subject. In the example of FIG. 18, image 313 shows the current index based on a scale of 100 for a well-balanced dietary index. When the user selects image 313, display device 30 displays dietary log data for calories, salinity, lipids etc., as illustrated in FIG. 5, on display 390. Thus, the user can view the subject's dietary information using display device 30.

Image 314 is, for example, an image for viewing a report on sleep of the subject. When the user selects image 314, display device 30 displays data on display 390 for sleep such as a sleep time as illustrated in FIG. 3. Thus, the user can view the subject's sleep information using display device 30.

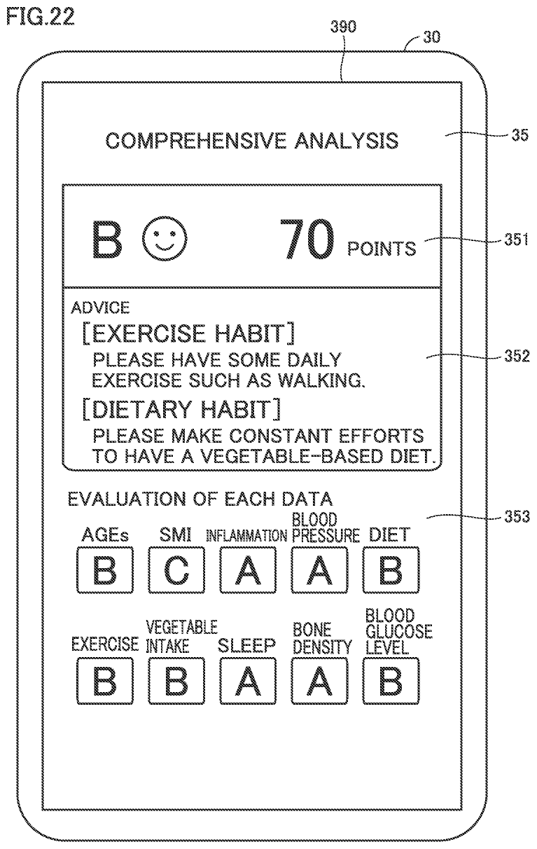

Image 315 is, for example, an image for viewing advice information. When the user selects image 315, then, as shown in FIG. 22, display device 30 displays a comprehensive analysis screen 35 on display 390 for viewing a comprehensive analysis result.

Comprehensive analysis screen 35 includes an image 351 indicating a score and an evaluation rank corresponding to the comprehensive analysis result, an image 352 indicating advice for at least one of a dietary habit, an exercising habit, a sleeping habit, and a mental health condition, and an image 353 indicating an evaluation result for each data of the subject. Advice device 50 calculates the score and evaluation rank corresponding to the comprehensive analysis result indicated in image 351 based on the evaluation result for each data of the subject shown in image 353. In doing so, when calculating the comprehensive analysis result, advice device 50 may apply predetermined weighting to each of the plurality of data of the subject. Thus, the user can view advice information provided to the subject using display device 30.

The viewing information shown in FIGS. 18 to 22 is an example, and may be changed for each subject based for example on the subject's age, gender, medical history, etc. The weighting applied in calculating the comprehensive analysis result may also be changed for each subject based on the subject's age, gender, medical history, etc.

### [Process by Advice Device]

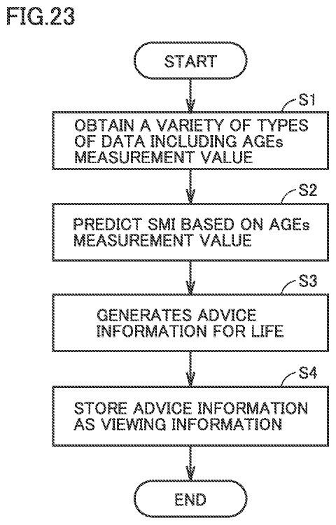

Advice device 50 performs a process, as will be described with reference to FIG. 23. FIG. 23 is a flowchart of a process performed by advice device 50 to provide advice according to an embodiment. The process includes steps (hereafter referred to as “S”) shown in FIG. 23, which are implemented by computing device 510 executing advice program 521.

As illustrated in FIG. 23, advice device 50 obtains an AGEs measurement value from AGEs measurement device 11 (S1). Advice device 50 predicts an SMI based on the obtained AGEs measurement value (S2).

Advice device 50 generates advice information based on the AGEs measurement value and at least one of a plurality of other data of the subject including the SMI prediction value (S3). Advice device 50 stores the advice information in storage device 520 as viewing information 523 (S4). Thus, advice device 50 can output the advice information stored as viewing information 523 to display device 30 in response to a request received from display device 30 to allow a user to view the advice information.

Thus, according to an embodiment, advice device 50 can generate advice information based on an AGEs measurement value and in addition thereto a plurality of other data of the subject that are different from the AGEs measurement value, and the advice device can thus provide the subject with advice optimal for reviewing a lifestyle habit. Furthermore, advice device 50 generates advice information based on an SMI predicted based on an AGEs measurement value, and even though SMI is slower than AGEs in presenting a result corresponding to a lifestyle habit, the advice device can provide the subject with optimal advice based on the SMI prediction value.

### [Aspects]

It will be understood by those skilled in the art that the exemplary embodiments described above are specific examples of the following aspects.

(Clause 1) According to one aspect, an advice device comprises a computing device and a communication device communicably connected to the computing device. The communication device is configured to obtain a measurement value of an advanced glycation end product of a subject and a plurality of other data of the subject that are different from the measurement value of the advanced glycation end product. The computing device is configured to generate advice information for a life of the subject based on the measurement value of the advanced glycation end product and at least one of the plurality of other data obtained by the communication device.

The advice device according to clause 1 can generate advice information based on an AGEs measurement value and in addition thereto a plurality of other data of the subject that are different from the AGEs measurement value, and the advice device can thus provide the subject with advice optimal for reviewing a lifestyle habit.

(Clause 2) The advice device according to clause 1, wherein the computing device is configured to generate the advice information based on the at least one other data obtained on a date correlated with a date on which the measurement value of the advanced glycation end product obtained by the communication device was obtained.

The advice device according to clause 2 generates the advice information based for example on at least one of the plurality of data of the subject that is obtained on a date closest to a date on which the AGEs measurement value is obtained, and the advice device can thus provide a user with advice which is accurate for reviewing a lifestyle habit.

(Clause 3) The advice device according to clause 1 or 2, wherein the communication device is configured to obtain the measurement value of the advanced glycation end product periodically at a first interval, and obtain specific data periodically at a second interval longer than the first interval, the specific data being included in the plurality of other data. The computing device is configured to obtain a result of predicting the specific data based on the specific data obtained by the communication device and a change in time series of the measurement value of the advanced glycation end product, and the computing device generates the advice information based on the result of predicting the specific data.

The advice device according to clause 3 predicts an SMI based for example on a measurement value of SMI, which is obtained at a longer interval than the AGEs measurement value, and a change in time series of the AGEs measurement value, and the advice device generates the advice information based on a result of predicting the SMI, and even though SMI is slower than AGEs in presenting a result corresponding to a lifestyle habit, the advice device can provide the user with optimal advice based on the SMI prediction value.

(Clause 4) The advice device according to clause 3, wherein an evaluation of the measurement value of the advanced glycation end product goes up and an evaluation of the specific data does not change, the computing device is configured to generate the advice information based on the result of predicting the specific data.

For example, when an evaluation of an AGEs measurement value goes up and an evaluation of an SMI does not change, the advice device according to clause 4 generates the advice information based on an SMI predicted based on the AGEs measurement value and can thus provide optimal advice to the user.

(Clause 5) The advice device according to clause 3, wherein when an evaluation of the measurement value of the advanced glycation end product goes down and an evaluation of the specific data does not change, the computing device is configured to generate the advice information based on the result of predicting the specific data.

For example, even when an evaluation of an AGEs measurement value goes down and an evaluation of an SMI does not change, the advice device according to clause 5 generates the advice information based on an SMI predicted based on the AGEs measurement value and can thus provide optimal advice to the user.

(Clause 6) The advice device according to any one of clauses 3 to 5, wherein the specific data is a measurement value of a skeletal muscle mass of the subject.

The advice device according to clause 6 can provide the user with optimal advice based on the SMI prediction value.

(Clause 7) The advice device according to any one of clauses 1 to 6, wherein the plurality of other data includes data of at least one of skeletal muscle mass, inflammation, blood pressure, diet, exercise, vegetable intake, sleep, bone density, blood glucose level, and stress of the subject.

The advice device according to clause 7 generates the advice information based on data of at least one of skeletal muscle mass, inflammation, blood pressure, diet, exercise, vegetable intake, sleep, bone density, blood glucose level, and stress of the subject in addition to the AGEs measurement value, and the advice device can thus provide the user with optimal advice for reviewing a lifestyle habit.

(Clause 8) The advice device according to any one of clauses 1 to 7, wherein the advice information includes advice for at least one of a dietary habit, an exercising habit, a sleeping habit, and a mental health condition of the subject.

The advice device according to clause 8 can provide the user with advice for at least one of a dietary habit, an exercising habit, a sleeping habit, and a mental health condition of the subject.

(Clause 9) The advice device according to any one of clauses 1 to 8, wherein when an evaluation of the measurement value of the advanced glycation end product goes up through a life following the advice information, the computing device is configured to generate new advice to include continuing the life following the advice information.

When the subject leads a life following the advice information and thus achieves an AGEs measurement value with an evaluation going up, the advice device according to clause 9 can advise the subject to continue to improve a lifestyle habit.

(Clause 10) The advice device according to any one of clauses 1 to 9, wherein when an evaluation of the measurement value of the advanced glycation end product does not go up through a life following the advice information, the computing device is configured to notify the subject of the advice information again.

When the subject leads a life following the advice information and still does not achieve an AGEs measurement value with an evaluation going up, the advice device according to clause 10 can provide the same advice information to the subject to cause the subject to improve a lifestyle habit.

(Clause 11) In one aspect, an advice method comprises: obtaining a measurement value of an advanced glycation end product of a subject and a plurality of other data of the subject that are different from the measurement value of the advanced glycation end product; and generating advice information for a life of the subject based on the measurement value of the advanced glycation end product and at least one of the plurality of other data obtained in the step of obtaining.

The advice method according to clause 11 can generate advice information based on an AGEs measurement value and in addition thereto a plurality of other data of the subject that are different from the AGEs measurement value, and the advice method can thus provide the subject with advice optimal for reviewing a lifestyle habit.

(Clause 12) In one aspect, an advice program causes a computing device to perform: obtaining a measurement value of an advanced glycation end product of a subject and a plurality of other data of the subject that are different from the measurement value of the advanced glycation end product; and generating advice information for a life of the subject based on the measurement value of the advanced glycation end product and at least one of the plurality of other data obtained in the step of obtaining.

The advice program according to clause 12 can generate advice information based on an AGEs measurement value and in addition thereto a plurality of other data of the subject that are different from the AGEs measurement value, and the advice program can thus provide the subject with advice optimal for reviewing a lifestyle habit.

(Clause 13) In one aspect, an advice system comprises: a measurement device that measures an advanced glycation end product of a subject; an advice device that generates advice information for a life of the subject based on a measurement value of the advanced glycation end product obtained from the measurement device and at least one of a plurality of other data of the subject that are different from the measurement value of the advanced glycation end product; and a display device that displays the advice information obtained from the advice device.

The advice system according to clause 13 can generate advice information based on an AGEs measurement value and in addition thereto a plurality of other data of the subject that are different from the AGEs measurement value, and the advice system can thus provide the subject with advice optimal for reviewing a lifestyle habit.

### REFERENCE SIGNS LIST

1 advice system, 11 AGEs measurement device, 12 body composition meter, 13 questionnaire, 14 sphygmomanometer, 15 dining hall, 16 activity meter, 17 vegetable intake measurement device, 18 sleep sensor, 19 bone densitometer, 20 continuous glucose monitoring device, 30 display device, 31 home screen, 32 AGEs viewing screen, 33 SMI viewing screen, 34 SMI prediction screen, 35 comprehensive analysis screen, 50 advice device, 311, 312, 313, 314, 315, 321, 322, 323, 331, 332, 333, 334, 341, 342, 351, 352, 353 image, 390 display, 510 computing device, 520 storage device, 521 advice program, 522 user identification information, 523 viewing information, 524 advice information, 530 communication device.

1. An advice device that provides life-related advice, comprising:
a computing device; and
a communication device communicatively connected to the computing device, wherein
the communication device is configured to obtain a measurement value of an advanced glycation end product of a subject and a plurality of other data of the subject that are different from the measurement value of the advanced glycation end product, and
the computing device is configured to generate advice information for a life of the subject based on the measurement value of the advanced glycation end product and at least one of the plurality of other data obtained by the communication device.

2. The advice device according to claim 1, wherein the computing device is configured to generate the advice information based on the at least one other data obtained on a date correlated with a date on which the measurement value of the advanced glycation end product obtained by the communication device was obtained.

3. The advice device according to claim 2, wherein
the communication device is configured to:
obtain the measurement value of the advanced glycation end product periodically at a first interval; and
obtain specific data periodically at a second interval longer than the first interval, the specific data being included in the plurality of other data, and
the computing device is configured to:
obtain a result of predicting the specific data based on the specific data obtained by the communication device and a change in time series of the measurement value of the advanced glycation end product; and
generate the advice information based on the result of predicting the specific data.

4. The advice device according to claim 3, wherein when an evaluation of the measurement value of the advanced glycation end product goes up and an evaluation of the specific data does not change, the computing device is configured to generate the advice information based on the result of predicting the specific data.

5. The advice device according to claim 3, wherein when an evaluation of the measurement value of the advanced glycation end product goes down and an evaluation of the specific data does not change, the computing device is configured to generate the advice information based on the result of predicting the specific data.

6. The advice device according to claim 3, wherein the specific data is a measurement value of a skeletal muscle mass of the subject.

7. The advice device according to claim 1, wherein the plurality of other data includes data of at least one of skeletal muscle mass, inflammation, blood pressure, diet, exercise, vegetable intake, sleep, bone density, blood glucose level, and stress of the subject.

8. The advice device according to claim 1, wherein the advice information includes advice for at least one of a dietary habit, an exercising habit, a sleeping habit, and a mental health condition of the subject.

9. The advice device according to claim 1, wherein when an evaluation of the measurement value of the advanced glycation end product goes up through a life following the advice information, the computing device is configured to generate new advice to include continuing the life following the advice information.

10. The advice device according to claim 1, wherein when an evaluation of the measurement value of the advanced glycation end product does not go up through a life following the advice information, the computing device is configured to notify the subject of the advice information again.

11. An advice method for providing life-related advice by a computing device, the advice method comprising:
obtaining a measurement value of an advanced glycation end product of a subject and a plurality of other data of the subject that are different from the measurement value of the advanced glycation end product; and
generating advice information for a life of the subject based on the measurement value of the advanced glycation end product and at least one of the plurality of other data obtained in the step of obtaining.

12. A non-transitory computer-readable storage medium storing an advice program that provides life-related advice, the advice program causing a computing device to perform:
obtaining a measurement value of an advanced glycation end product of a subject and a plurality of other data of the subject that are different from the measurement value of the advanced glycation end product; and
generating advice information for a life of the subject based on the measurement value of the advanced glycation end product and at least one of the plurality of other data obtained in the step of obtaining.

13. An advice system that provides life-related advice, comprising:
a measurement device that measures an advanced glycation end product of a subject;
an advice device that generates advice information for a life of the subject based on a measurement value of the advanced glycation end product obtained from the measurement device and at least one of a plurality of other data of the subject that are different from the measurement value of the advanced glycation end product; and
a display device that displays the advice information obtained from the advice device.
